(19) United States
(12) Patent Application Publication
(10) Pub. No.: US 2025/0312777 A1
(43) Pub. Date: Oct. 9, 2025

(54) CUBIC FLUORITE RARE-EARTH HIGH ENTROPY OXIDES AND THEIR CATALYSIS APPLICATIONS

(71) Applicant: Khalifa University of Science and Technology, Abu Dhabi (AE)

(72) Inventor: Kyriaki POLYCHRONOPOULOU, Abu Dhabi (AE)

(21) Appl. No.: 18/866,274

(22) PCT Filed: Apr. 25, 2023
(86) PCT No.: PCT/IB2023/054256

§ 371 Date: Nov. 15, 2024

Related U.S. Application Data
(60) Provisional application No. 63/342,238, filed on May 16, 2022.

(51) Int. Cl. B01J 23/83; B01J 23/10; B01J 37/02; B01J 37/03; B01J 37/08; C01B 3/40; C07C 1/12

(52) U.S. Cl. CPC B01J 23/83; B01J 23/10; B01J 37/0201; B01J 37/0236; B01J 37/038; B01J 37/08; C01B 3/40; C07C 1/12; C01B 2203/0238; C01B 2203/1058

(57) ABSTRACT

In general, the embodiments of the present disclosure describe Ceria-based mixed metal high entropy oxide (HEO) catalysts, namely CeLaPrSmGdO, its Nickel supported counterpart catalysts for use in water gas shift reaction and methods of making Ceria based mixed metal high entropy oxide catalysts and Nickel supported mixed metal high entropy oxide catalysts.

### CROSS-REFERENCE TO RELATED APPLICATION

This application claims benefit of U.S. Provisional Application No. 63/342,238 filed on May 16, 2022. U.S. Provisional Application No. 63/342,238 is incorporated herein by reference. A claim of priority is made.

### BACKGROUND

The present disclosure relates to Ceria-based mixed metal high entropy oxide (HEO) catalysts, namely CeLaPrSmGdO, its Ni supported counterpart catalysts and methods of synthesizing the same.

Power to gas technology that depends on CO₂ hydrogenation to methane is considered as one of the most promising processes that utilizes CO₂ as a precursor to convert it into valuable hydrocarbons that serve both the industrial and the environmental fields. Operating CO₂ methanation reaction is usually favored over metal supported catalysts due to the kinetic and thermodynamic limitations; the challenge is to reduce the thermodynamically stable CO₂ molecule (ΔH°₂₉₈ₖ=−393.5 KJ/mol) to CH₄. All these classes of catalysts were found to exhibit improved selectivity towards CH₄, enhanced activity and high stability under time on stream. Among all, supported metals, either transition (TM) and noble metals (NM), experienced high catalytic activity for converting CO₂ into methane. Compared to the noble metals (e.g.: Pd, Pt, Ir, Rh, Re, and Ru), transition metals (e.g., Co, Fe, Ni and Mg) are known for their competitive cost and high activity for the reaction at hand. Both activity and selectivity orders of these metals were classified as follows: Ru>Fe>Ni>Co>Rh>Pd>Pt and Pd>Pt>Ir>Ni>Rh>Co>Fe>Ru, respectively.

Coupling the Ni beneficial features for the CO₂ activation with the concept of the High Entropy Oxide structure can lead to a new class of catalysts with interesting properties to be explored. The High Entropy Oxides (HEO) and Entropy Stabilized Oxides (ESO) are a new category of materials that was explored in 2015 by Rost et al.; both classes of materials can be considered as the natural evolution in the development of catalysts. The HEO can be considered analogous thermodynamically to the High Entropy Alloys (HEA).

### SUMMARY

In general, embodiments of the present disclosure describe Ceria-based mixed metal high entropy oxides (HEO), namely CeLaPrSmGdO, its Ni supported counterpart catalysts and methods of synthesizing the same. In particular, the embodiments of the present disclosure describe Ceria-based mixed metal high entropy oxide (HEO) catalysts facilitating the water gas shift reaction comprising an HEO catalyst being of the form MxOy where M represents a group of at least 5 different oxide-forming metallic cations or dopants, x represents the number of metal cations (M) or atoms and y represents the number of oxygen anion (O) or atoms, wherein the HEO catalyst that maintains single phase composition.

Embodiments of the present disclosure further describe a Nickel supported HEO catalyst facilitating the water gas shift reaction, wherein the catalyst comprises of Ceria-based mixed metal high entropy oxides (HEO) and at least one transition metal.

Embodiments of the present disclosure describe a method of making a Ceria-based mixed metal high entropy oxide (HEO) catalyst facilitating the water gas shift reaction, comprising dissolving the precursor salts of dopants and coprecipitating the HEO as a slurry mixture. This is followed by drying the slurry mixture. The dried slurry mixture is further calcined and ground, sufficient to form the HEO support.

Embodiments of the present disclosure also describe a method of making a Ceria-based mixed metal high entropy oxide (HEO) catalyst facilitating the water gas shift reaction, comprising physically mixing the precursor salts of dopants and dry ball milling the physically mixed dopants. The method further comprises calcining the dry ball milled mixture and grinding the calcined powder sufficient to form the HEO catalyst or HEO support.

Embodiments of the present disclosure further describe a method of making a Nickel supported Ceria-based mixed metal high entropy oxide (HEO) catalyst facilitating the water gas shift reaction, comprising dispersing coprecipitated HEO supports in water followed by wet impregnation of Nickel onto the HEO supports. The water is then evaporated. This is followed by calcining the nickel impregnated HEO supports.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 is a flowchart illustrating the steps utilized in co-precipitation method for preparation of Ceria-based mixed metal high entropy oxide (HEO) catalyst, according to some embodiments.

FIG. 2 is a flowchart illustrating the steps utilized in dry ball milling method for preparation of Ceria-based mixed metal high entropy oxide (HEO) catalyst, according to some embodiments.

FIG. 3 is a flowchart illustrating the steps utilized in the method for preparation of Nickel supported Ceria-based mixed metal high entropy oxide (HEO) catalyst, according to some embodiments.

FIG. 4(A) shows the X-ray diffraction of the multi-elemental system (CeLaPrSmGd)O which was synthesized using two different methods: dry ball milling (DBM) and co-precipitation (CP).

FIG. 4(B) shows the X-ray diffraction patterns of the effect of calcination duration (4 h vs. 14 h) on the HEO structure formed by Dry Ball Milling.

FIG. 4(C) shows the X-ray diffraction patterns of the BM systems, following ball milling for 4, 8, and 12 h.

FIG. 4(D) shows the X-ray diffraction patterns of the Ni supported HEO catalysts with varying Ni loading (5, 10, 15 wt. %).

FIG. 5(A) shows the HRTEM characterization carried out over the 15Ni-500/HEO-CP catalyst.

FIG. 5(B) shows the Selected Area Electron Diffraction (SAED) pattern of the reduced 15Ni-500/HEO-CP catalyst.

FIG. 6(A) shows the High resolution HAADF-STEM image of the reduced 15Ni-500/HEO-CP.

FIG. 6(B-H) show the High resolution HAADF-STEM image of elemental mapping of the reduced 15Ni-500/HEO-CP. FIG. 6(B) shows the image of Ce mapping. FIG. 6(C) shows the image of La mapping. FIG. 6(D) shows the image of Pr mapping. FIG. 6(E) shows the image of Ni mapping. FIG. 6(F) shows the image of O mapping. FIG. 6(G) shows the image of Gd mapping. FIG. 6(H) shows the image of Sm mapping.

FIG. 7 shows the H₂-TPD profiles of the 5Ni-500/HEO-CP, 10Ni-500/HEO-CP, 15Ni-500/HEO-CP and 10Ni-900/HEO-CP catalysts.

FIG. 8(A-D) show the catalytic performance of the HEO based catalysts: FIG. 8(A) shows the CO₂ Conversion, FIG. 8(B) shows the CH₄ Selectivity, FIG. 8(C) shows the CO Selectivity and FIG. 8(D) shows the CH₄ Yield.

FIG. 9(A-D) shows the catalytic performance as a function of time of the HEO based catalysts. FIG. 9(A) shows the CO₂ Conversion, FIG. 9(B) shows the CH₄ Selectivity, FIG. 9(C) shows the CO Selectivity and FIG. 9(D) shows the CH₄ Yield.

FIGS. 10(A-E) show the Bright field HRTEM image (FIG. 10(A)), HAADF-STEM and RGB image of La—O—Ni (FIGS. 10(B-C)), SAED pattern (FIG. 10(D)) and EELS survey spectrum (FIG. 10(E)) of the spent 15% Ni-500/HEO-CP.

FIG. 11(A-C) show DRM activity of Ni-HEO catalysts: FIG. 11(A) shows CH₄ conversion rate, FIG. 11(B) shows CO₂ conversion rate, and FIG. 11(C) shows H₂:CO product ratio. Reaction conditions are: Temperature: 750° C., Pressure: 1 atm, Gas feed: 50,000 mL·gcat⁻¹·h⁻¹; 1:1:2 molar ratio of CH₄:CO₂:He. The catalysts were reduced prior to DRM activity in 20% H₂ atmosphere for 1 h. Ni-HEO: 5% Ni on high entropy oxide support; Ni-HEO-4DBM: 5% Ni on HEO using 4 h dry ball milling; Ni-HEO-4DBM: 5% Ni on HEO using 12 h dry ball milling.

FIGS. 12(A-D) show the TPO profile of spent catalyst Ni-HEO (FIG. 12(B)), Ni-HEO_4h DBM (FIG. 12(C)) and Ni-HEO_12h DBM (FIG. 12(D)) and all the catalysts combined in (FIG. 12(A)), after 24 hours of DRM.

### DETAILED DESCRIPTION

The present disclosure relates to Ceria-based mixed metal high entropy oxides (HEO), namely CeLaPrSmGdO, its Ni supported counterpart catalysts and methods of synthesizing the same. In particular, the embodiments of the present disclosure describe Ceria-based mixed metal high entropy oxide (HEO) catalysts facilitating the water gas shift reaction. Some embodiments of the present disclosure describe an HEO catalyst being of the form MxOy where M represents a group of at least 5 different oxide-forming metallic cations or dopants, x represents the number of metal cations (M) or atoms and y represents the number of oxygen anion (O) or atoms. Yet other embodiments of the present disclosure describe an HEO catalyst that maintains single phase composition at high operating temperatures. Other embodiments of the present disclosure describe an HEO catalyst that maintains single phase composition from room temperature to 900° C.

In certain embodiments of the present disclosure, the dopants are rare earth metals. In yet other embodiments the dopants are elements selected from Cerium (Ce), Lanthanum (La), Praseodymium (Pr), Samarium (Sm), Gadolinium (Gd). In some embodiments of the present disclosure, the dopants are added in equimolar amounts.

Embodiments of the present disclosure describe a Nickel supported HEO catalyst facilitating the water gas shift reaction wherein the catalyst comprises of Ceria-based high entropy oxides (HEO) and at least one transition metal. Some embodiments of the present disclosure describe the above catalyst wherein the HEO is used as a support and the transition metal is Nickel. In certain other embodiments of the present disclosure the nickel is impregnated onto the HEO support.

### Methods for Preparation of Ceria-Based Mixed Metal High Entropy Oxide (HEO) Catalysts

### Method for Preparation of HEO Catalysts Using Co-Precipitation

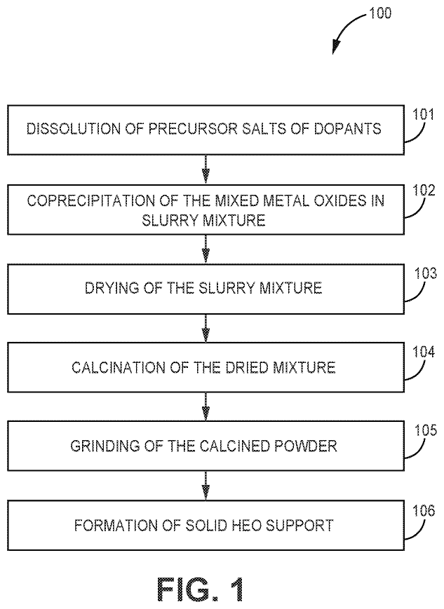

FIG. 1 is a flowchart illustrating the steps utilized in a method for preparation of Ceria-based mixed metal high entropy oxide (HEO) catalyst facilitating the water gas shift reaction, according to one or more embodiments of the present disclosure. As shown in FIG. 1, the method may comprise dissolving (101) the precursor salts of dopants followed by coprecipitating (102) the HEO as a slurry mixture and drying (103) of the slurry mixture. The dried slurry mixture is then calcined (104) followed by grinding (105) the calcined powder sufficient to form the HEO support.

The step 101 includes dissolving equimolar quantities of the precursors salts of the rare earth metals or dopants in the present disclosure, namely lanthanum (III) nitrate hexahydrate (La(NO₃)₃·6H₂O Sigma Aldrich >99.0%), cerium (III) nitrate hexahydrate (Ce(NO₃)₃·6H₂O Sigma Aldrich 99.0%), praseodymium (III) nitrate hexahydrate (Pr(NO₃)₃·6H₂O Sigma Aldrich 99.0%), gadolinium (III) nitrate hexahydrate (Gd(NO₃)₃·6H₂O Sigma Aldrich >99.0%) and samarium (III) nitrate hexahydrate (Sm(NO₃)₃·6H₂O Sigma Aldrich >99.0%), in 30 ml of distilled water and stirring continuously at 65° C. and 340 rpm for 15 hours.

The step 102 involves the co-precipitation of the mixed metal oxides by adding 2.5 ml of ammonium hydroxide solution to the precursor salts solution followed by continuous stirring for 2 hours 65° C. and 340 rpm. The co-precipitation was carried out in different amounts of ammonium hydroxide solution ranging from 1.5 ml to 3.5 ml of ammonium hydroxide solution. The optimum precipitation was obtained at 2.5 ml of ammonium hydroxide solution. Different temperature ranges were used to determine the optimum precipitation. A temperature range of about 45° C. to about 80° C. was used. The optimum precipitation was observed to be at 65° C. The range of rpm used was from 270 rpm to about 450 rpm. The optimum precipitation was observed at 340 rpm. Similarly, the time range was stirring was determined by changing the stirring time from about 1.5 hours to 3.0 hours. The optimum precipitation was obtained at continuous stirring for 2 hours. A slurry mixture comprising the co-precipitated dopants was obtained.

The step 103 comprises drying of the slurry mixture obtained in step 102. The slurry mixture thus obtained in step 102 was subjected into overnight drying at 60° C. in step 103.

The step 104 comprises calcination of the dried slurry mixture. The dried material was calcined (in step 104) at 900° C. for 4 hours at 5° C./min. Different temperatures, calcination time and rate for calcination were used to standardize the results for optimal conditions. For optimizing the conditions for calcination, a temperature range of 450° C. to about 950° C. were used. The time range used was between 2.5 hours and 5.5 hours. The rate of calcination was chosen from about 2° C./min to about 6° C./min. The process of calcination of the dried slurry mixture was optimized at the rate of 5° C./min at the temperature of 900° C. for 4 hours.

In step 105, the calcined powder was collected and ground using an agate mortar.

In step 106, the solid “CeGdLaPrSmO” support was formed following the above process steps and was labelled as HEO-CP.

### Method for Preparation of HEO Catalysts Using Dry Ball Milling

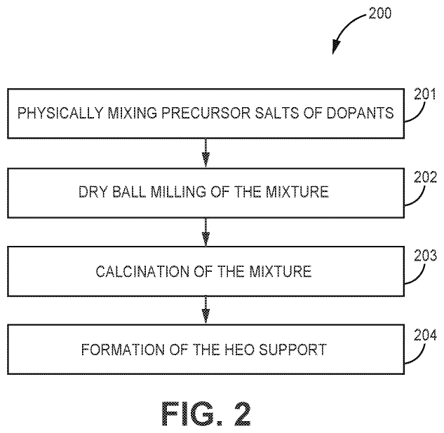

FIG. 2 is a flowchart illustrating the steps utilized in another method for preparation of Ceria-based mixed metal high entropy oxide (HEO) catalysts facilitating the water gas shift reaction, according to one or more embodiments of the present disclosure. As shown in FIG. 2, the method may comprise physically mixing (201) the precursor salts of the dopants, followed by dry ball milling (202) of the physically mixed dopants. The calcination (203) of the dry ball milled mixture was performed and the “CeGdLaPrSmO” support catalyst was formed (204).

The step 201 includes physically mixing equimolar quantities of lanthanum (III) nitrate hexahydrate (La(NO₃)₃·6H₂O Sigma Aldrich >99.0%), cerium (III) nitrate hexahydrate (Ce(NO₃)₃·6H₂O Sigma Aldrich 99.0%), praseodymium (III) nitrate hexahydrate (Pr(NO₃)₃·6H₂O Sigma Aldrich 99.0%), gadolinium (III) nitrate hexahydrate (Gd(NO₃)₃·6H₂O Sigma Aldrich >99.0%) and samarium (III) nitrate hexahydrate (Sm(NO₃)₃·6H₂O Sigma Aldrich >99.0%), followed by dry ball milling (BM).

The step 202 includes the dry ball milling of the physically mixed dopants. The physically mixed dopants obtained from step 201 were dry ball milled for 4, 8 and 12 hours at a speed of 250 rpm. A range of rpm between 200 rpm to about 300 rpm was used to ascertain the optimal synthesis conditions. The duration of ball milling is another parameter to explore towards its effect on homogenizing the crystal structure. To do so the ball milling duration was increased at increments of 4 hours. As explained later, the diffraction patterns of the ball milled systems show clear hump shaped peaks as a proof of the secondary phases or impurities.

In step 203, the calcination (203) of the dry ball milled mixture was performed The BM materials were calcined at 900° C. for 4 hours at a rate of 5° C./min. Different temperatures, calcination time and rate for calcination were used to standardize the results for optimal conditions. For optimizing the conditions for calcination, a temperature range of 450° C. to about 950° C. were used. The time range used was between 2.5 hours and 5.5 hours. The rate of calcination was chosen from about 2° C./min to about 6° C./min. The process of calcination of the BM systems was optimized at the rate of 5° C./min at the temperature of 900° C. for 4 hours.

The step 204 comprises the formation of the “CeGdLaPrSmO” support catalyst. The “CeGdLaPrSmO” support material formed were labelled as HEO-4BM, HEO-8BM and HEO-12BM. The high entropy oxide (HEO) obtained when the ball milling was done for the duration of 4 hours was labelled as HEO-4BM. The high entropy oxide (HEO) obtained when the ball milling was done for the duration of 8 hours was labelled as HEO-8BM. The high entropy oxide (HEO) obtained when the ball milling was done for the duration of 12 hours was labelled as HEO-12BM.

### Method of Preparation of Nickel Supported HEO Catalysts

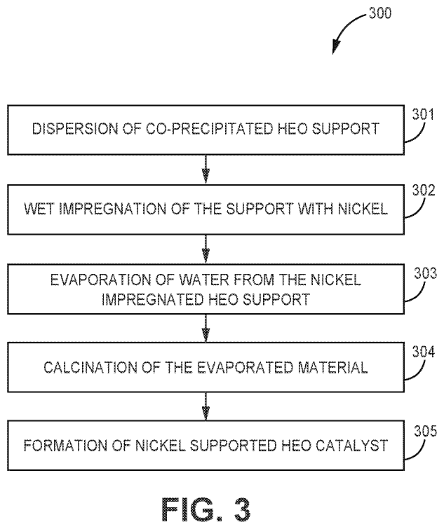

FIG. 3 is a flowchart illustrating the steps utilized in a method for preparation of Nickel (Ni) supported Ceria-based mixed metal high entropy oxide (HEO) catalyst facilitating the water gas shift reaction. As shown in FIG. 3, the method may comprise dispersing (301) coprecipitated HEO supports formed by the co-precipitation method described above. The dispersion of HEO supports in water was then followed by wet impregnation (302) of Nickel onto the HEO supports. This was proceeded by the evaporation (303) of water from the nickel impregnated HEO supports. Calcination (304) of the nickel impregnated HEO supports was done to form (305) the nickel supported HEO catalyst.

In step 301, the coprecipitated HEO supports were dispersed separately in 30 ml of distilled water. The co-precipitated HEO supports were prepared by dissolving equimolar quantities of the precursors salts of the rare earth metals or dopants in the present disclosure, namely lanthanum (III) nitrate hexahydrate (La(NO₃)₃·6H₂O Sigma Aldrich >99.0%), cerium (III) nitrate hexahydrate (Ce(NO₃)₃·6H₂O Sigma Aldrich 99.0%), praseodymium (III) nitrate hexahydrate (Pr(NO₃)₃·6H₂O Sigma Aldrich 99.0%), gadolinium (III) nitrate hexahydrate (Gd(NO₃)₃·6H₂O Sigma Aldrich >99.0%) and samarium (III) nitrate hexahydrate (Sm(NO₃)₃·6H₂O Sigma Aldrich >99.0%), in 30 ml of distilled water and stirring continuously at 65° C. and 340 rpm for 15 hours. As stated above in step 102, the co-precipitation of the mixed metal oxides was carried out by adding 2.5 ml of ammonium hydroxide solution to the precursor salts solution followed by continuous stirring for 2 hours 65° C. and 340 rpm. The slurry mixture thus obtained was subjected into overnight drying at 60° C. The dried material was calcined (as in step 104) at 900° C. for 4 hours at 5° C./min. the calcined powder was collected and ground using an agate mortar (as in step 105). The solid “CeGdLaPrSmO” support was formed (as in step 106) following the above process and was labelled as HEO-CP.

This was followed by step 302, which involves the wet impregnation of nickel onto the HEO supports. In step 302, the desired amount of Ni(NO₃)₃·6H₂O (Sigma Aldrich >99.0%) was dissolved in 10 ml of distilled water then added slowly on each of the supports.

In step 303, the nickel impregnated HEO supports were subjected to continuous stirring at 65° C. until the water evaporated. The catalysts were labeled as follows: 10% Ni-900/HEO-4BM, 5% Ni-500/HEO-CP, 10% Ni-500/HEO-CP, 10% Ni-900/HEO-CP and 15% Ni-500/HEO-CP.

In step 304, calcination of the evaporated material was carried out. Among the catalysts prepared, the catalysts labeled as 10%/Ni-900/HEO-4BM and 10% Ni-900/HEO-CP were calcined after the Ni impregnation at 900° C. for 4 hours at a rate of 5° C./min, whereas the 5% Ni-500/HEO-CP, 10% Ni-500/HEO-CP and 15% Ni-500/HEO-CP were calcined at 500° C. for 5 hours at a rate of 2.6° C./min. The step 305 shows the formation of Ni supported HEO catalyst after the calcination in step 304 . . . . As stated above, the catalysts were labeled as follows: 10% Ni-900/HEO-4BM, 5% Ni-500/HEO-CP, 10% Ni-500/HEO-CP, 10% Ni-900/HEO-CP and 15% Ni-500/HEO-CP.

One or more embodiments of the HEO catalyst formed by the methods explained above have single phase crystalline structure. Some embodiments of the present disclosure describe single phase HEO catalyst which have cubic fluorite crystalline structure. The methods described above also give rise to embodiments that form single phase HEO catalyst which maintains phase composition, without transformation, from room temperature to the operating range of the catalyst. Other embodiments of the present disclosure describe an HEO catalyst that maintains single phase composition from room temperature to 900° C.

### Effect of Synthesis Method on the HEO Structure

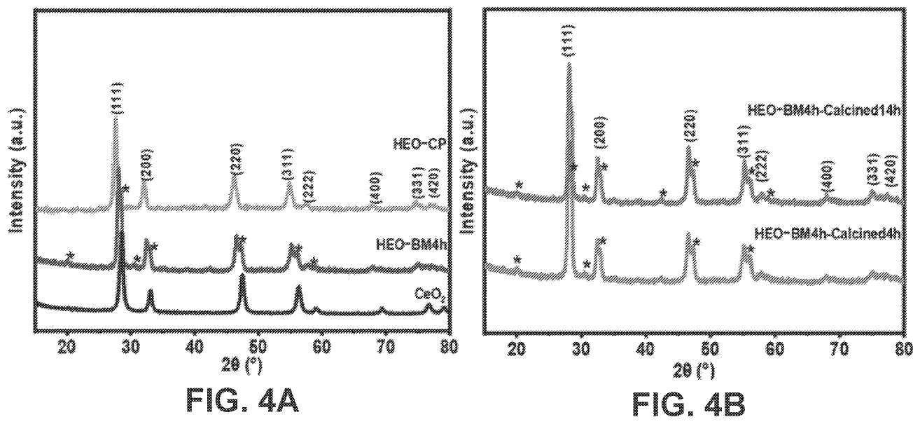

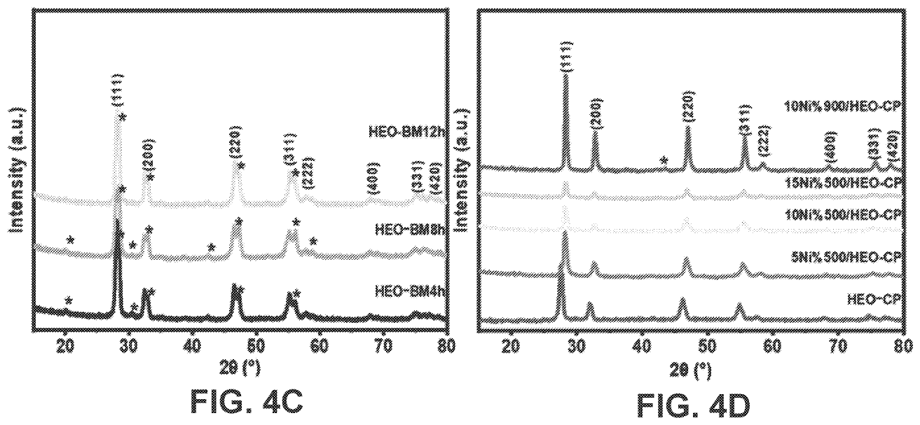

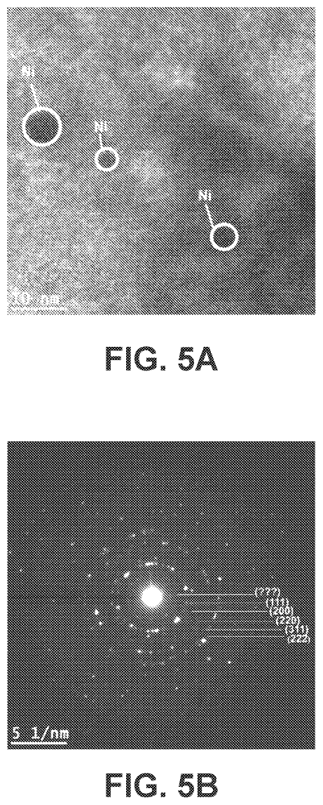

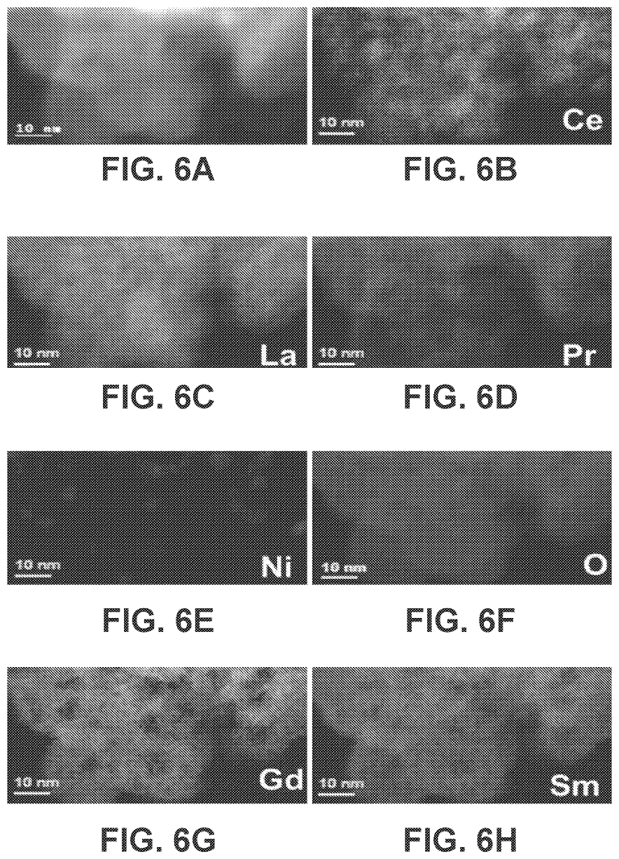

X-ray diffraction (XRD) was used to investigate the crystallinity of the HEO support in comparison to the binary reference materials (Ce20MO-CP) (SI) as well as the Ni supported on the HEO catalysts. It should be mentioned here that it is highly possible for nanodomains of hetero-phases to be formed but not be traced using XRD due to the multi-elemental composition of the support. FIG. 4(A) depicts the X-ray diffraction of the multi-elemental system (CeLaPrSmGd)O which was synthesized using two different methods, dry ball milling (DBM) and co-precipitation (CP), following calcination at 900° C. for 4 h with a 5° C./min ramp rate. Based on the XRD patterns, it was determined that only the CP system crystalized into single phase with fluorite lattice symmetry (Fm-3m), with an evidence of forming a rather homogeneous solid solution indicated by the detected sharp peaks, such as the reflections at 2θ=28.56°, 33.01°, 47.46°, 56.28°, 59.22°, 69.46°, 76.69° and 79.12° corresponding to (111), (200), (220), (311), (222), (400), (331) and (420) planes of cubic fluorite (ceria-related phase), respectively. In this case, it can be stated that CP succeeded to give rise to single phase HEO structure. It can be clearly seen that the above-mentioned peaks had a slight shift towards lower angles of diffraction compared to pure CeO₂ diffraction pattern which has the following reflections from planes (111), (200), (220), (311), (222), (400), (331) and (420) that give rise to diffraction peaks at 2θ=28.21°, 32.66°, 46.85°, 55.53°, 58.19°, 68.46°, 75.41° and 77.77°. The observed shift can be linked to the lattice parameter distortion (expansion) which is caused by the addition of the different dopants with varying cationic radii as shown in Table 1. On the other hand, the ball milled support gave rise to diffraction peaks having obvious humps on the lowering side of the main ceria (cubic structure)-related peaks, such as in the 2θ=28.4° and 32.9° denoted in FIG. 4(A); this leads to the conclusion that the lattice did not crystalize into single fluorite structure, whereas some phase separation and/or impurity phases can be present.

TABLE 1
Textural properties of the HEO support,
and the HEO-CP supported Ni catalysts

| | BET | Pore | HEO | HEO Lattice |
|---|---|---|---|---|
| Oxide | Surface | Volume | Crystallite size | parameter |
| System | Area (m²/g) | (cm³/g) | (nm)* | (Å) |
| HEO-CP | 3.3 | 0.02 | 17.0 | 0.55762 |
| 10Ni-900/ | 1.9 (3.1) | 0.03 (0.03) | 26.4 (17.2) | 0.54651 |
| HEO-CP | | | | |
| 5Ni-500/ | 2.9 (2.7) | 0.02 (0.02) | 15.0 (14.7) | 0.54905 |
| HEO-CP | | | | |
| 10Ni500/ | 3.8 (3.5) | 0.02 (0.02) | 16.1 (14.1) | 0.54829 |
| HEO-CP | | | | |
| 15Ni-500/ | 3.0 (2.9) | 0.02 (0.02) | 19.3 (12.2) | 0.54727 |
| HEO-CP | | | (35)^{†} | |

*By using the Scherrer equation at the fluorite structure predominant peak of (111) reflection.
*Values in parenthesis refer to the spent catalysts.
^{†}This value originates from the HRTEM studies.

Though this finding needs more investigation (e.g., identifying the hetero-phase nanodomain) using sensitive techniques, (e.g. HRTEM), and will be discussed below. Based on the XRD results, the co-precipitation method led to the successful co-crystallization of all the metal cations into a single phase; this can be due to the almost similar precipitation rate values of the different metal hydroxide species (Ksp of Ce(OH)₃ 2×10-20, Ksp of La(OH)₃ 2×10-21, Ksp of Pr(OH)₃ 3.39×10-24, Ksp of Gd(OH)₃ 2.8×10-23). Similar precipitation rates act as the driving force to bring all the cations into the same lattice and overcome the sluggish diffusion phenomena, whereas kinetics of growth and nucleation role in the co-precipitation cannot be neglected. During the co-precipitation and the following calcination, the different metal cations are competing to occupy the same site in the lattice at the same time. On the contrary, under dry ball milling conditions (BM), the metal nitrate precursors are being subjected into mechanical forces. However, based on the XRD findings, it seems that the stress introduced was not adequate to break the pre-existing bonds and assist into the formation of new ones leading to a uniform structure (same crystal lattice) of all the competing cations. In the case of CP, the mediation of the hydroxide phase (the cations precipitate as M(OH)x) seems to be crucial for such required cations proximity. Each of the metal salts used as precursors respond to the applied stress differently as their M-NO₃ bond has different enthalpy of formation and thermogravimetric decomposition profiles.

### Effect of Calcination Conditions (Temperature (T), Duration (D) on the HEO Structure of BM Solids

As mentioned above, it was rather challenging to achieve single phase formation (HEO) following the BM synthesis. Given the pivotal role of the treatment/calcination temperature in the achievement of HEO structure, the calcination time was increased from 4 h to 14 h maintaining the temperature at 900° C. The effect of calcination duration (4 h vs. 14 h) on the structure, is shown in FIG. 4(B). It was found that the increase of the thermal treatment duration by 10 hours could not homogenize the multi-phases received under ball milling conditions (hump-shape peaks due to phase separation can still be noticed). Since high temperature did not contribute to the uniformity of the structure for the given elements, it might mean that there is another driving force for doing so. Clearly, the elements selected for the rare earth system determined the response of the material to the thermal treatment.

Another parameter to explore regarding the effect on homogenizing the crystal structure of rare earth oxides towards forming the HEO was the duration (time) of the BM. This was done by increasing the ball milling time at increments of 4 h. The diffraction patterns of the BM systems, following ball milling for 4, 8, and 12 h, are shown in FIG. 4(C) where clear hump-shaped peaks can be seen as verification of the secondary phases (impurities). It must be mentioned here that there are reports in the literature mentioning 30-40 hours of planetary ball milling as the appropriate synthesis condition towards single phase formation for HEA (high entropy alloy), but such a scenario is far from the energy saving process that is attempted to developed in the context of the present disclosure.

### Effect of Synthesis Method on the Ni Supported HEO Structure

From the above analysis, it is obvious that only CP method gave rise to HEO ‘real’ structure. Therefore, the HEO were used as supports for the preparation of the Ni supported catalysts. FIG. 4(D) presents the diffraction patterns of the Ni supported catalysts with varying Ni loading (5, 10, 15 wt. %), and with the support being calcined at two different temperatures (50° C. vs. 900° C.); namely, the XRD profiles of 10Ni-900/HEO-CP, 5Ni-500/HEO-CP, 10Ni-500/HEO-CP and 15Ni-500/HEO-CP catalysts are presented in order to evaluate the effect of: (a) Ni metal loading (wt. %) and (b) calcination temperature (500° C. vs. 900° C.) on the HEO catalyst structure. It is important to note that in all the Ni supported catalysts, regardless of the metal loading, no NiO phase was found in the XRD patterns, with the exemption of the 10% Ni-900/HEO-CP catalyst. For the latter, the existence of a peak at 2θ=43.5° corresponding to NIO FCC (200) plane was found. For the rest of the catalysts, where NiO could not be detected, nm-sized crystallites (higher Ni dispersion) can be assumed, escaping the XRD detection. In addition, all the patterns prove the presence of uniform fluorite crystal structure. It was observed that Ni addition caused a slight shift of the XRD diffraction angles to higher diffraction angles values compared to their values in the case of the HEO support alone.

As shown in FIGS. 4(A-D), the fluorite (CeO2-related) (111) diffraction peak is the predominant one, and it corresponds to the lowest surface energy according to the literature. The HEO crystallite size was calculated using Scherrer's equation and the values are listed in Table 2 and Table 3. Comparing the values in Tables 2 and 3 (binary oxides as reference), it can be noted that as we move from the binary (reference) to the 5-elements HEO system, a significant reduction in the crystallite size occurs, in agreement with the solid solution formation according to the literature. For example, in the case of the HEO-CP support the crystallite size was 49.1%, 66.6% and 53.4% smaller than the size obtained in the case of Ce-20PrO-CP, Ce-20GdO-CP and Ce-20LaO-CP, respectively. In the case of the Ni catalysts, the Ni crystallite size is increasing due to the successive calcination steps at high temperatures following the Ni addition (impregnation) onto the support.

TABLE 2
Surface atomic composition as derived using XPS

| | 5Ni500/ | 10Ni500/ | 15Ni500/ | 10Ni900/ |
|---|---|---|---|---|
| | HEO-CP | HEO-CP | HEO-CP | HEO-CP |
| Element | Atomic (%) | Atomic (%) | Atomic (%) | Atomic (%) |
| Ce3d | 0.2 | 0.8 | 0.7 | 1.4 |
| Gd3d | 1.3 | 4.0 | 4.3 | 3.5 |
| La3d5 | 1.4 | 3.4 | 2.9 | 4.6 |
| Pr3d | 1.8 | 5.4 | 5.2 | 4.7 |
| Sm3d | 1.5 | 5.0 | 5.4 | 4.3 |
| Ni3P | 1.4 | 5.4 | 5.2 | 3.1 |

TABLE 3
Textural properties of the rare-earth
oxide binary reference supports

| Oxide | BET Surface Area | Pore Volume | Crystallite size |
|---|---|---|---|
| System | (m²/g) | (cm³/g) | (nm)¹ |
| Ce—20PrO-CP | 8.4 | 0.06 | 34.6 |
| Ce—20GdO-CP | 0.1 | 0.05 | 51.9 |
| Ce—20LaO-CP | 8.3 | 0.05 | 17.7 |

### Analyzing the Structure of Ni Supported HEO Catalyst Using High Resolution Transmission Electron Microscopy (HRTEM)

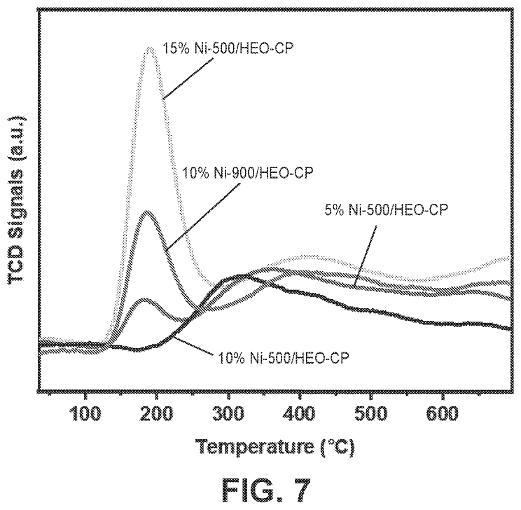

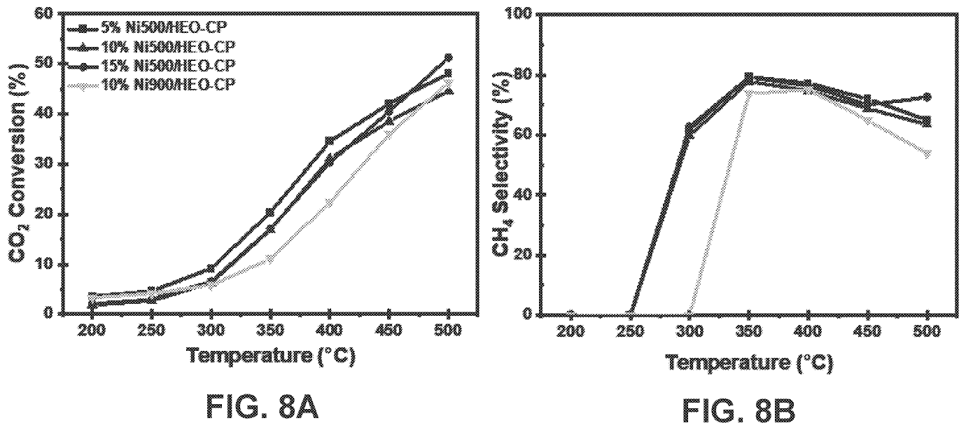

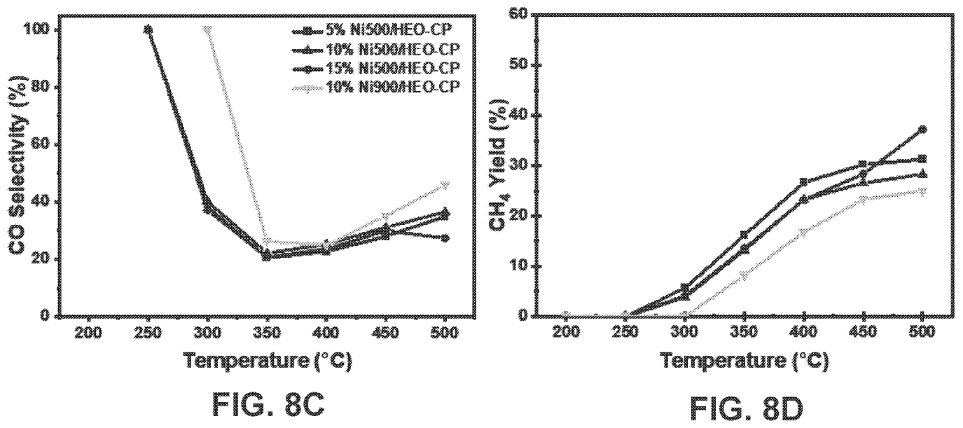

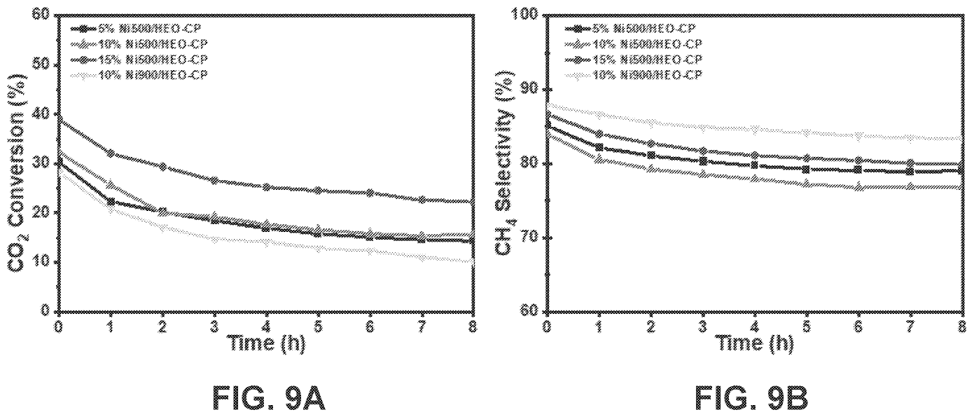

Based on the XRD results (FIG. 4(D)), presented earlier, the catalysts calcined at 500° C. showed no NiO diffraction peaks most likely due to the possibility of having the Ni particles finely distributed (below 5 nm), thus escaping the detection ability of the XRD. To verify the above hypothesis, HR-TEM characterization was carried out over the 15Ni-500/HEO-CP catalyst (highest Ni loading) as probe catalyst; the HRTEM was collected following its reduction at 500° C. for 2 h. The nickel particles, as shown in FIG. 5(A), are distributed in a variety of sizes that ranged between 3-6 nm on the support. By further analyzing the HRTEM image, the poly-crystallinity of the catalyst could be indicated as depicted in the FIG. 5(A). The Selected Area Electron Diffraction (SAED) pattern is shown in FIG. 5(B) and additionally verifies the crystallinity of the reduced 15Ni-500/HEO-CP catalyst through the observed rings/spots structure formed. The SAED pattern also shows that the material is mainly crystallized in the fluorite (Fm-3m) phase and the inter-planar spacings were found to be 0.318 nm, 0.277 nm, 0.195 nm, 0.165 nm and 0.161 nm, 0.133 nm, 0.125 nm, 0.122 nm and 0.111 nm which are in good agreement with the literature values corresponding to the (111), (200), (220), (311), (222), (400), (331), (420) and (422) planes of fluorite lattice, respectively. FIGS. 6(A-H) illustrates the High Angle Annular Dark Field-Scanning Transmission Electron Microscopy (HAADF-STEM) image and the elemental mapping of the reduced 15Ni-500/HEO-CP catalyst, where Ni is clearly seen to be distributed on top of the support without evidence of any aggregation/clustering. It is noteworthy to mention that in the Gd mapping (FIG. 6(G)), it is seen that Gd encircles/encapsulates the Ni particles, which is not the case in the rest of the elements.

### H₂-TPD Chemisorption and Ni Dispersion

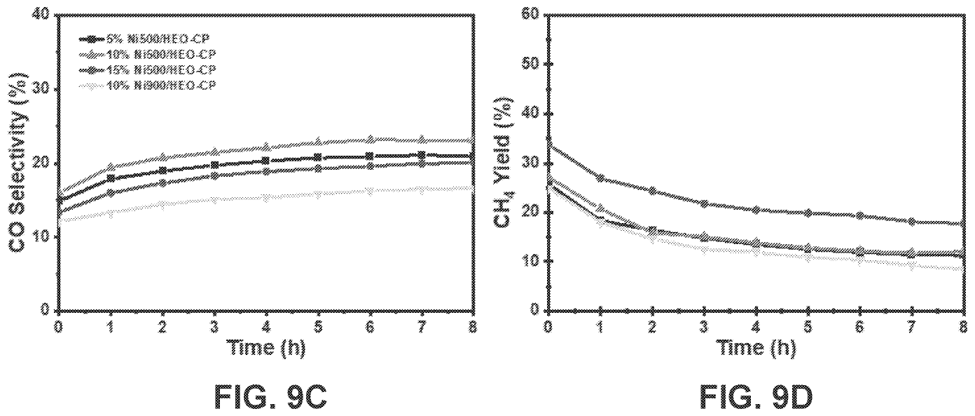

In order to investigate the dispersion of the Ni catalysts, H₂ chemisorption followed by temperature programmed desorption (TPD) experiments were performed. FIG. 7 shows the H₂ desorption traces obtained over the Ni catalysts of interest. Almost all the desorption profiles present similar characteristics, namely a main peak at ˜180° C. followed by a second wider peak with Tₘₐₓ at around 350-400° C.; these peaks can be assigned to the desorption of hydrogen from the free (loosely bound to the support) Ni crystallites, and the Ni crystallites in strong interaction with the HEO support. Based on the amount of the hydrogen desorbed and considering a H/Ni_{surf} ratio of 1/1 the Ni dispersion can be calculated for the catalysts. In particular, Ni dispersions of 40%, 32%, 22%, 21%, 20% were measured for the 5Ni/HEO, 10Ni/HEO, 15Ni/HEO and 10Ni900/HEO catalysts, respectively. A mean Ni particle size (dNi, nm) of 2.5, 3.1, 4.4. and 4.7 was estimated based on the H₂ chemisorption studies. The Ni crystallite size based on the H₂ chemisorption studies is in good agreement with the HRTEM studies previously presented. Only the 10Ni/HEO catalyst presents a different trace (shape and position of the curve) demonstrating the impact of the Ni loading and thus Ni crystallite size to the formulation of the Ni-support interface. Interestingly, the 10Ni/HEO catalyst does not present a low temperature peak at around 180° C. as the rest of the catalysts. Comparing the H₂ desorption profiles of the two Ni catalysts with the same metal loading (10 wt. %) where the HEO support has been subjected into different calcination temperature, namely 500° C. vs. 900° C., it can be concluded that the HEO500 smaller crystallite size, (16 nm) facilitates strong metal support interaction (SMSI) with Ni as compared to the case of the HEO900 support (26.4 nm); the latter is reflected to the first H₂ desorption peak appearing at higher temperature in the case of the particular catalyst. The SMSI effects play a critical role in the CO₂ methanation reaction as discussed previously.

### EXAMPLES

Two routes of decarbonization through CO₂ Catalytic Conversion are presented below:

### Example 1: CO₂ Methanation Reaction Activity

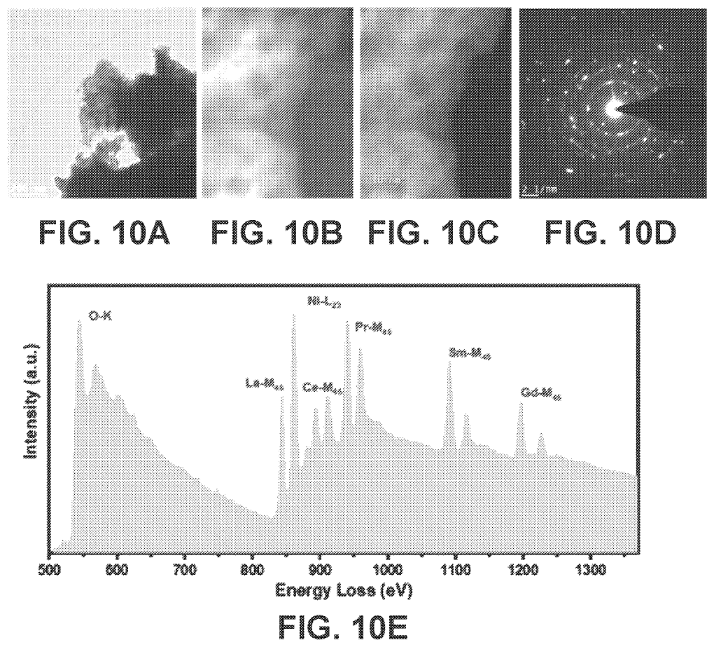

The Ni supported catalysts were evaluated for the CO₂ methanation reaction and their activity was expressed in terms of CO₂ conversion, (XCO₂, %). The reproducibility of the catalytic experiments was ensured by repeating the experiments at least three times. CH₄ and CO are the only reaction products found and the carbon balance was found to present minor deviations (˜3%). The side product (CO) can be produced through Reverse Water Gas Shift (RWGS) reaction (CO₂+H₂χCO+H₂O, ΔH°₂₉₈ₖ=+41 KJ/mol) and often leads to the deterioration of the yield of the main product (CH₄). FIGS. 8(A-D), present the temperature effect on the CO₂ conversion (X_{CO2}, %), CH₄ and CO selectivity (S_{CH4}, S_{CO}, %) and CH₄ yield (Y_{CH4}, %), respectively, obtained over the Ni catalysts supported on HEO. As can be seen in FIG. 8(A), the X_{CO2} rises as the temperature increases from 200° C. to 500° C. for all the catalysts studied. Highest performance is presented in the case of 15% Ni-500/HEO-CP catalyst at 500° C. (50% CO₂ conversion), whereas the 5% Ni-500/HEO-CP catalyst experienced better activity compared to the rest catalysts in the range of 200° C.<T<500° C., as can be seen by the general increasing trend of CO₂ conversion. It is also important to note that despite the exothermicity of the CO₂ methanation reaction (at T>600° C., the ΔG of methanation reaction turns to positive values), the X_{CO2} is maintaining an uprising trend up to 500° C. FIGS. 8(B-C) illustrate the selectivity of the desired product (CH₄) and undesired product (CO), respectively. The maximum recorded S_{CH4} (%) was achieved in the case of 5% Ni-500/HEO-CP, 10Ni-500/HEO-CP and 15% Ni-500/HEO-CP at 350° C. and it was found to be 80% for all of the catalytic systems, while at temperatures higher than 350° C., all the catalysts exhibited a clear decrease in the S_{CH4}, whereas the CO selectivity starts taking over (FIG. 8(C)). This can be understood on the basis of the competition of the endothermicity (ΔH°₂₉₈ₖ=+41 KJ/mol) of the RWGS reaction with the exothermicity of the CO₂ methanation reaction (ΔH°₂₉₈ₖ=−165 KJ/mol. The S_{CH4} drop with the concomitant S_{CO} increase is more pronounced in the case of 10Ni/900HEO catalyst. FIG. 8(D) shows the Y_{CH4} for all the Ni catalysts. It is obvious that calcination at 900° C. leads to severe nickel sintering and ultimately the catalyst presents the lowest performance in the whole temperature window. The results are in agreement with the trends observed in literature.

### Analysis of Catalytic Stability in the CO₂ Methanation Reaction

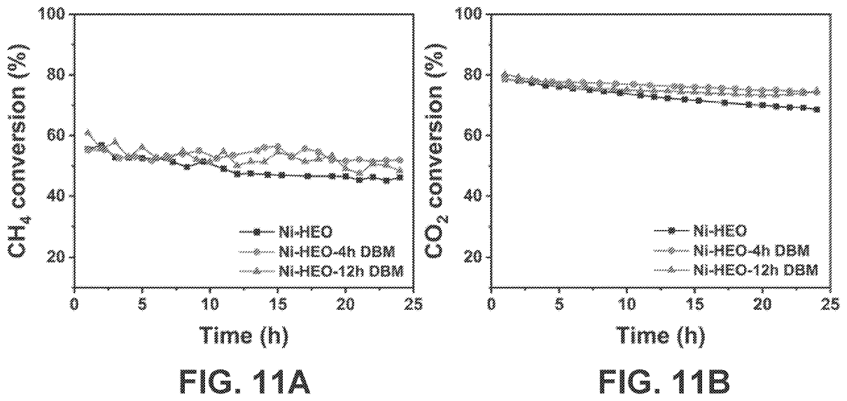

FIGS. 9(A-D) demonstrate the performance of the HEO catalysts with time on stream at 400° C. for 8 h. A general decrease trend in terms of the CO₂ conversion (FIG. 9(A)), CH₄ yield (FIG. 9(D)), and CH₄ selectivity (FIG. 9(B)),) was noted to occur after the first hour followed by a rather stable catalytic performance for the rest of the test (8 h). In addition, the S_{CO} for the four catalysts was noticed to increase after 1 h on stream and then maintain a rather stable profile during the 8 hours (FIG. 9(C)). The best catalytic performance for the 15Ni-500/HEO and 10Ni-500/HEO catalysts, in terms of activity and selectivity, can be further explained through the Ni particles, which are responsible for the activation and dissociation of H₂. Literature provides that smaller Ni particles are more selective towards CH₄ production. According to the H₂-TPD studies presented earlier in the present disclosure, the 10Ni-500/HEO and 15Ni-500/HEO catalysts presented Ni dispersion of 23 and 21%, respectively. Raman studies demonstrated the presence of oxygen vacancies in this catalyst too; the latter is responsible for the CO₂ adsorption onto the support, as previously shown in the CO₂-TPD. The present disclosure describes a first demonstration to utilize HEOs as supports for Ni for the CO₂ methanation reaction. Therefore, it will require more optimization in terms of the textural properties to tune the selectivity of CH₄ and maximize it.

### Spent Catalyst Characterization

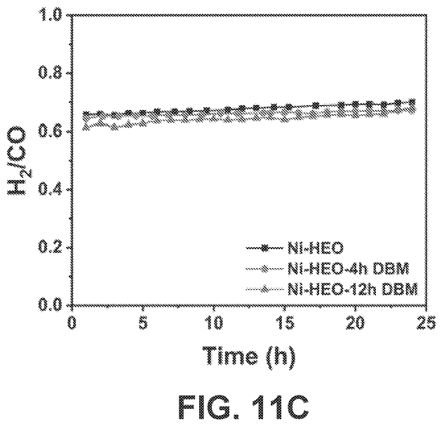

FIGS. 10(A-E) show the HRTEM analysis for the 15Ni-500/HEO catalyst following the CO₂ methanation reaction (8 hours on stream). The Bright Field HRTEM image for the spent catalyst (FIG. 10(A)) shows the existence of carbon which verifies the latter findings for the slight drop in the performance of the catalyst during the CO₂ methanation. HAADF-STEM mapping (FIG. 10(B-C)) using the color coding: R: Red: La, G: Green: O and B: Blue: Ni shows some color combinations that can be explained as phases formed by the chemical interaction of the primary phases, such as the orange (RG mixing) and the cyan (GB mixing). The SAED pattern (FIG. 10(D)) is in agreement with the XRD pattern of the used material, where it still holds its polycrystallinity. FIG. 10(E) shows the Electron Energy Loss Spectroscopy (EELS) survey spectrum of the spent 15% Ni-500/HEO-CP.

### Example 2: Dry Reforming of Methane (DRM)

Dry reforming of methane (DRM) is a unique reaction which consumes two greenhouse gases: methane (CH₄) and carbon dioxide (CO₂); and converts into useful synthesis gas (equation 1).

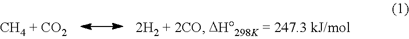

Nickel (Ni) metal well known for its capability of catalytically activating the C—H bond, however, is highly prone to deactivation due to coking and sintering. To solve the issues, Ni atoms anchored on different supports are used as DRM catalysts. Among various supports, lanthanide-based materials are highly preferable for the DRM reaction due to their alkaline nature and are highly active for CO₂ adsorption. On the other hand, since the introduction of high entropy materials, the high entropy oxides (HEO) are emerging as interesting support materials for different catalytic reactions due to the exhibition of high stability, more defects, oxygen vacancies, and lattice distortions at elevated temperatures which are helpful to achieve high catalytic performance.

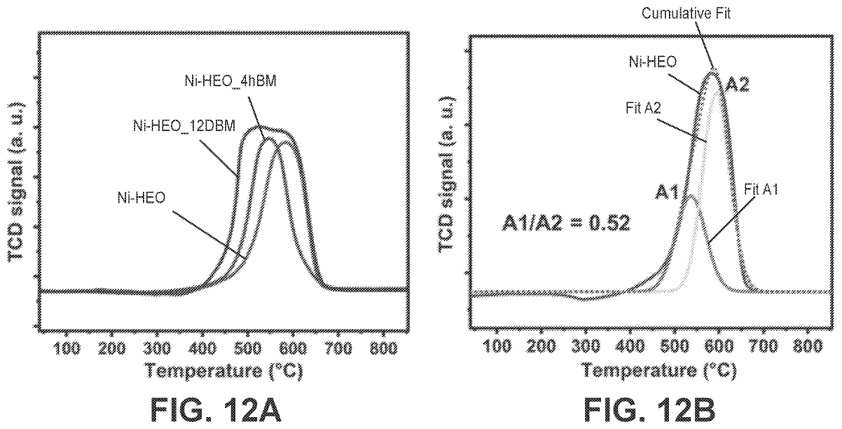

In the present disclosure, Ni supported HEO catalysts were synthesized using wet-chemical (Ni-HEO) and mechanochemical (dry ball milling (DBM) for 4 h and 12h time; Ni-HEO-4h DBM and Ni-HEO-12h) methods. The developed catalysts were tested for the DRM reaction and the results are given in FIG. 11(A-C). The DBM catalysts have shown relatively high CH₄ (FIG. 11(A)) and CO₂ (FIG. 11(B)) conversion rates as compared to Ni-HEO catalyst. This might be due to the production of more defects or oxygen vacancies during the mechanical forces exerted in the support during the ball milling process which helps in better conversion rates. From FIG. 11(A-B), it is evident that in all the catalysts, the CO₂ conversion rates are higher than CH₄ conversion rates and also the H₂/CO product ratio is much lower than 1 (FIG. 11(C)). This is due to prevailing of reverse water gas shift reaction (RWGS, equation 2), a side reaction occurring simultaneously on the catalyst at elevated temperatures.

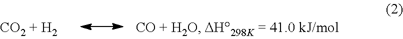

The RWGS reaction consume CO₂ and convert into CO, hence higher CO₂ conversion rates and decrement in H₂/CO product ratio. Although nominal CH₄ and CO₂ conversion rates are observed with the current catalysts, the constant H₂/CO product ratio throughout the reaction is interesting. On the other hand, we observed coking in all the catalysts after the DRM reaction. The coke formation can happen via methane cracking (equation 3).

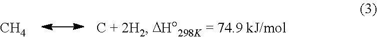

Moreover, the high CO₂ conversion rates could be due to the possible Boudouard reaction (equation 4) happening at the catalyst surface, where the accumulated carbon reacts with CO₂ and converts into CO.

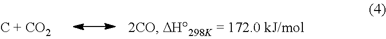

Overall, the HEO based catalysts have shown good CH₄ and CO₂ conversion rates and a stable H₂/CO product ratio. It is believed that the methane cracking and Boudouard reactions limit a high DRM activity. A further modification in the composition of the catalyst or in the synthesis methodology is expected to address the above issues during the DRM reaction.
Coking Studies after DRM

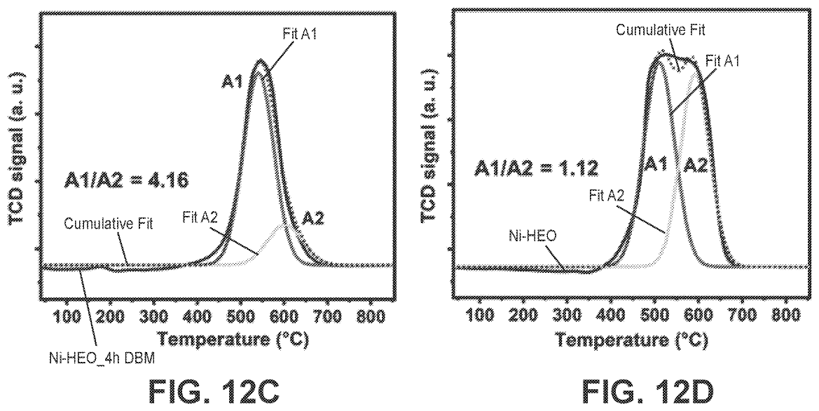

FIG. 12(A-D) present the temperature programmed oxidation (TPO) profiles for the spent catalysts after 24 h of DRM reaction. In the plots for FIG. 12(A-D), A1 and A2 correspond to the Disordered structured carbons, such as carbon nanofibers and carbon nanotubes (500-600° C.), and to the Graphitic carbon (600-800° C.), respectively. The formation of Graphitic carbon is less in the case of Ni-HEO_4h DBM sample as compared to Ni-HEO and Ni-HEO_12h DBM proving the beneficial role of the DBM process into the DRM anti-coking.

While the disclosure has been described with reference to an exemplary embodiment(s), it will be understood by those skilled in the art that various changes may be made, and equivalents may be substituted for elements thereof without departing from the scope of the embodiment(s). In addition, many modifications may be made to adapt a particular situation or material to the teachings of the embodiment(s) without departing from the essential scope thereof. Therefore, it is intended that the disclosure is not limited to the disclosed embodiment(s), but that the disclosure will include all embodiments falling within the scope of the appended claims. Various examples have been described. These and other examples are within the scope of the following claims. Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus, the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto.

Various examples have been described. These and other examples are within the scope of the following claims.

1-32. (canceled)

33. A catalyst, the catalyst comprising:
a mixed metal high entropy oxide (HEO) including a formula MₓO_{y}, where M represents a group of at least 5 different oxide-forming metallic cations or dopants, x represents a number of metal cations (M) or atoms, y represents a number of oxygen anion (O) or atoms, and the HEO maintains phase composition.

34. The catalyst of claim 33, wherein M includes one or more rare earth metals.

35. The catalyst of claim 33, wherein M includes Cerium (Ce), Lanthanum (La), Praseodymium (Pr), Samarium (Sm), and Gadolinium (Gd).

36. The catalyst of claim 33, wherein the dopants are added in equimolar amounts.

37. The catalyst of claim 33, wherein the catalyst includes a formula: CeLaPrSmGdO.

38. The catalyst of claim 33, wherein the HEO is used as a support and the catalyst further includes a transition metal.

39. The catalyst of claim 38, wherein the transition metal includes Nickel.

40. The catalyst of claim 39, wherein the Nickel is impregnated onto the support.

41. A method of making a catalyst, the method comprising:
dissolving precursor salts of dopants;
coprecipitating to form a slurry mixture;
drying the slurry mixture; and
calcining the dried slurry mixture to form a mixed metal high entropy oxide (HEO).

42. The method of claim 41, wherein the precursor salts include one or more of lanthanum (III) nitrate hexahydrate, cerium (III) nitrate hexahydrate, praseodymium (III) nitrate hexahydrate, gadolinium (III) nitrate hexahydrate, and samarium (III) nitrate hexahydrate.

43. The method of claim 41, wherein the precursor salts are dissolved in equimolar quantities.

44. The method of claim 41, wherein the precursor salts include one or more rare-earth metal salts.

45. The method of claim 41, wherein coprecipitating includes utilizing ammonium hydroxide.

46. The method of claim 41, wherein coprecipitating is performed at a temperature ranging from about 45° C. to about 80° C.

47. The method of claim 41, wherein the mixed metal high entropy oxide includes a formula CeLaPrSmGdO.

48. The method of claim 41, wherein the mixed metal high entropy oxide is in a form of a single phase.

49. A method of making a catalyst, the method comprising:
dispersing a coprecipitated ceria-based mixed metal high entropy oxide (HEO) support in water;
impregnating Nickel on the coprecipitated ceria-based mixed metal high entropy oxide (HEO) support to form a first product;
evaporating the water; and
calcining the first product to form a second product.

50. The method of claim 49, wherein the coprecipitated ceria-based mixed metal high entropy oxide (HEO) support includes a formula MₓO_{y}, where M represents a group of at least 5 different oxide-forming metallic cations or dopants, x represents a number of metal cations (M) or atoms, y represents a number of oxygen anion (O) or atoms, and the HEO maintains phase composition.

51. The method of claim 49, wherein the coprecipitated ceria-based mixed metal high entropy oxide (HEO) support includes a formula CeLaPrSmGdO.

52. The method of claim 49, wherein the Nickel provided for impregnation is in a form of Ni(NO₃)₃·6H₂O.
